(19) United States
(12) Patent Application Publication
(10) Pub. No.: US 2006/0111356 A1
(43) Pub. Date: May 25, 2006

(54) External preparation for allergic diseases

(75) Inventors: Akira Yagi, Fukuoka (JP); Takao Shida, Tokyo (JP); Kokushin Ryu, Tokyo (JP); Taiichi Kaku, Tokyo (JP); Yoshiharu Kadota, Osaka (JP)

Correspondence Address: BIRCH STEWART KOLASCH & BIRCH, PO BOX 747, FALLS CHURCH, VA, 22040-0747, US

(73) Assignee: Japan Bioproducts Inc. Co., Ltd., Tokyo (JP)

(21) Appl. No.: 10/512,096

(22) PCT Filed: Apr. 21, 2003
(86) PCT No.: PCT/JP03/05063

§ 371 Date: Dec. 27, 2005

(30) Foreign Application Priority Data
Apr. 22, 2002 (JP) 2002-119475

(51) Int. Cl. A61K 31/498

(52) U.S. Cl. 514/249

(57) ABSTRACT

The invention presents an external preparation for allergic diseases containing 3′-hydroxymethyl-4-hydroxypyrrolido[1,2-f]2′,5′-pipera-zinedione as an active ingredient. This compound has a prophylactic and therapeutic effect of allergic diseases, and the external preparation of the invention can be used in prophylaxis and treatment of various allergic diseases (for example, atopic dermatitis, urticaria, allergic rhinitis, allergic conjunctivitis, pollinosis, allergic asthma, and others).

### TECHNICAL FIELD

The present invention relates to an external preparation for allergic diseases. More particularly, it relates to an external preparation for allergic diseases containing a piperazinedione derivative as an active ingredient.

### BACKGROUND ART

Allergic diseases of type I such as atopic dermatitis, urticaria, allergic asthma and pollinosis are increasing every year, and effects are not limited to physical pain for patients, but are posing serious social problems accompanied by mental pain of patients and families. Causes of increase of such allergic diseases include, aside from genetic predisposition, changes in eating habit, changes in life style such as dwellings habitable for mite antigen and indoor rearing of pet animals, stress, air pollution, passive smoking, and other environmental changes, but clear conclusion or countermeasure is not obtained.

Allergic diseases of type I such as atopic dermatitis are induced by reaction of invading allergen and immunoglobulin E in the body of patient, stimulation of mast cells, and release of inflammatory mediator such as histamine or leukotriene. More specifically, when the allergen is bonded and crosslinked with bimolecular Fab of IgE antigen bonded with Fc portion on the cell surface of mast cell at the site of invasion, degranulation reaction occurs in the cell. As a result, histamine, leukotriene, serotonin, heparin, slow reacting substance of anaphylaxis (SRS-A), eosinotactic factor (ECF-A) and others in the granules are released, and a series of immunopharmacological reactions comprising smooth muscle contraction, mucus secretion promotion and vascular transmission promotion takes place, and allergic symptoms appear.

For treatment of such allergic diseases, steroids and vegetable extracts have been used, and steroids are preferred from the viewpoint of efficacy. Steroids are, however, accompanied by strong side effects and are not suited to long-term use. Further, sufficient effects may not be obtained if the method or period of use is not proper, and it has been rather difficult to use.

Thus, steroids hitherto used in treatment of allergic diseases have various problems, and it has been long desired to develop external preparations for allergic diseases small in side effects and easily usable for a long period.

The present inventors have isolated and purified human placental hydrolyzate in order to identify bioactive substances in human placental hydrolyzate, and discovered hydroxyproline derivatives having cell growth action and cell protection action, and filed a patent application (see WO99/47546 publication). The inventors have further promoted studies on the hydroxyproline derivatives, and found that a piperazinedione derivative is effective for preventing and treating allergic diseases, and completed the present invention.

The invention is based on such findings, and it is hence an object thereof to present an external preparation for allergic diseases containing a piperazinedione derivative as an active ingredient.

### DISCLOSURE OF THE INVENTION

The invention relates to an external preparation for allergic diseases containing 3′-hydroxymethyl-4-hydroxypyrrolido[1,2-f]2′,5′-piperazinedione derivative as an active ingredient.

The external preparation for allergic diseases of the invention is preferably used as a remedy for atopic dermatitis.

### BRIEF DESCRIPTION OF THE DRAWINGS

FIG. 1 is a diagram showing an immediate dermatitis onset suppressing effect of the compound contained as an active ingredient of the external preparation of the invention. In the diagram, ** denotes p<0.01.

### DETAILED DESCRIPTION OF THE PREFERRED EMBODIMENTS

3′-hydroxymethyl-4-hydroxypyrrolido[1,2-f]2′,5′-piperazinedione as the active ingredient of the external preparation of the invention is a known compound, which is represented by the following formula (1).

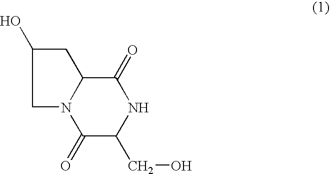

This compound can be obtained by the method disclosed, for example, in WO99/47546 publication mentioned above.

The compound of formula (1) has asymmetric carbon and cyclic structure in its molecule, and the compound of the invention includes all of optical isomers and geometrical isomers based on such asymmetric carbon and cyclic structure, and their mixtures. A preferred compound is, for example, cyclo (trans)-4-L-hydroxyprolyl-L-serine.

Applicable diseases of the external preparation for allergic diseases of the invention include, for example, atopic dermatitis, urticaria, allergic rhinitis, allergic conjunctivitis, pollinosis, and allergic asthma, or mainly allergic diseases of type I.

The external preparation of the invention may be mixed with proper pharmacologically required components such as pharmacologically acceptable additives (for example, carrier, excipient, diluent, etc.), and pharmacologically manufactured in various dosage forms of external preparation (ointment, liquid, lotion, spray, inhalant, nasal drops, eye drops, bath salts, etc.), and administered externally (topically) or by inhalation or aspiration. This preparation can be manufactured by conventional pharmaceutical means. The content of the compound of formula (1) can be properly changed depending on the dosage form, disease, or symptom of patient, and an effective amount of the compound of formula (1) is contained in the preparation.

An effective dose and administration schedule of the preparation of the invention can be determined empirically, which is known to those skilled in the art. The dose is adjusted properly depending on the administration route, symptom, or body weight and age of patient, and is selected in a range of 1 to 100 mg/kg body weight, and a preferred range is 2.5 to 50 mg/kg body weight, and more preferably about 25 mg/kg body weight, which is administered once a day or in several divided portions.

### INDUSTRIAL APPLICABILITY

The compound of formula (1) which is the active ingredient of the external preparation of the invention has prophylactic and therapeutic effect of allergic diseases, and the external preparation of the invention can be used in prophylaxis and treatment of various allergic diseases.

### EXAMPLES

The invention is more specifically described below by referring to Examples, but it must be noted that the invention is not limited to these Examples alone.

### Example 1

Test by Using Immediate Dermal Reaction Model

The experiment was conducted by using mice (BALB/cAnNCrj, female, 7 weeks of age, weighing 20 to 30 g).

Animals were passively sensitized by injecting diluted antiserum (10 μg/site) into the inside of auricle of mouse once a week for 3 weeks (a total of 3 times).

Allergic dermatitis was induced by injecting antigen (egg albumin/Evans blue), 48 hours after passive sensitization, by 0.5 mg/mouse (0.25 ml/mouse) into the caudal vein.

Test substances were prepared by manufacturing ointments from the compound of formula (1) by containing 9 mg/g (test drug 1), 3 mg/g (test drug 2), and 1 mg/g (test drug 3). The test drug was administered twice, 30 minutes and 60 minutes before induction, by applying 20 mg ointment/site to the inside skin of the auricle.

Dermatitis suppressant effect was evaluated by leak pigment measurement. More specifically, 30 minutes after the induction, the mouse was sacrificed by dislocation of cervical vertebrae, and both auricles were sampled. A pair of auricles were put in1N sodium hydroxide solution for 16 hours at 37° C., and tissue dissolved solution was obtained. In this solution, 2.5N phosphoric acid solution/acetone mixed solution (3:17) was added by 4 times of 1N potassium hydroxide solution, and stirred and centrifuged (3000 rpm, 15 minutes), and a supernatant was sampled. In the sampled supernatant, absorbance at 620 nm was measured, and the leak pigment amount was calculated. As control, a commercial drug, Tacrolimus, was used and tested similarly.

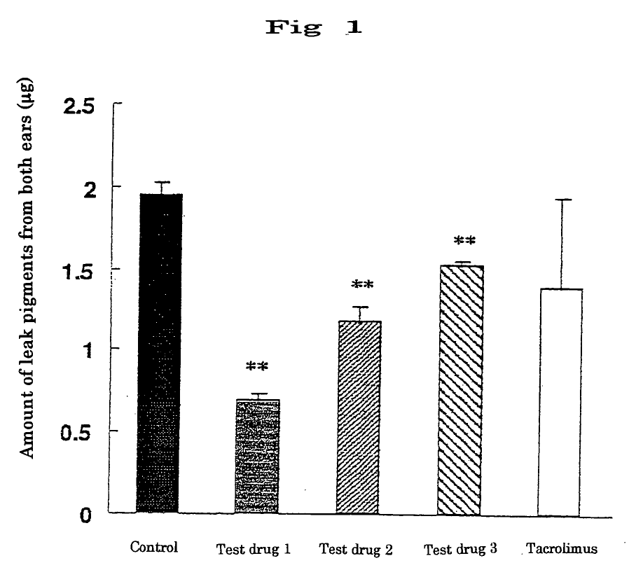

Results are shown in FIG. 1. As shown in FIG. 1, the test drug strongly suppressed leak of pigment, and was proved to prevent onset of dermatitis.

### Pharmaceutical Example 1

Preparation of Ointment

The compound of formula (1) was added to a hydrophilic ointment base mainly composed of vaseline, stearyl alcohol, propylene glycol, and polyoxyethylene hardened castor oil, and an ointment containing the compound by 9 mg/g was prepared by a conventional method.

1. An external preparation for allergic diseases containing 3′-hydroxy-methyl-4-hydroxypyrrolido[1,2-f]2′,5′-piperazinedione as an active ingredient.

2. The external preparation for allergic diseases of claim 1, wherein the preparation is a remedy for atopic dermatitis.

3. A method for treating allergic disease comprising administering an effective amount of 3′-hydroxymethyl-4-hydroxypyrrolido[1,2-f]2′,5′-piperazinedione.

4. The method of claim 3, wherein the allergic disease is atopic dermatitis.

5. A use of 3′-hydroxymethyl-4-hydroxypyrrolido[1,2-f]2′,5′-piperazinedione for manufacturing an external preparation for allergic diseases.

6. The use of claim 5, wherein the external preparation is any one of ointment, liquid, lotion, spray, inhalant, nasal drops, eye drops, and bath salts.
